(19) United States
(12) Patent Application Publication
(10) Pub. No.: US 2006/0073189 A1
(43) Pub. Date: Apr. 6, 2006

(54) Chewing gums, lozenges, candies, tablets, liquids, and sprays for efficient delivery of medications and dietary supplements

(75) Inventors: John M. Pinney, Bethesda, MD (US); Jack E. Henningfield, Baltimore, MD (US); L. David Taylor, Washington, DC (US); Edward J. Cone, Severna Park, MD (US)

Correspondence Address: BERENATO, WHITE & STAVISH, LLC, 6550 ROCK SPRING DRIVE, SUITE 240, BETHESDA, MD, 20817, US

(73) Assignee: NPD, LLC

(21) Appl. No.: 11/177,380

(22) Filed: Jul. 11, 2005

Related U.S. Application Data
(63) Continuation of application No. 09/573,982, filed on May 19, 2000, now abandoned.

(51) Int. Cl. A61K 9/68

(52) U.S. Cl. 424/440; 424/48

(57) ABSTRACT

The present invention relates to gums, lozenges, candies, tablets, liquids and spray compositions that contain orally administered medications and dietary supplements that are released in the oral cavity. The medications and dietary supplements contained therein may be delivered in a multi-phase mode. The compositions may also contain buffer systems that facilitate oral absorption. A rapid release is followed by slower release of medicant(s) and dietary supplements. The buffer system is released simultaneously with the medicant(s) and dietary supplements, thereby facilitating transmucosal and buccal absorption of active ingredient(s). The invention delivers, first, rapidly an initial pharmacologically effective dose of medicine and dietary supplements and, second, a prolonged pharmacologically sufficient dose for longer-term relief of symptoms or provision of therapeutic effect.

### BACKGROUND OF THE INVENTION

1. Field of the Invention

The present invention relates to gums, lozenges, candies, tablets, liquids and sprays; and more particularly, to chewing gum, lozenge, candy, tablet, liquid and spray compositions that contain orally administered medications and dietary supplements that are released in the oral cavity. The medications and dietary supplements contained in the gums, lozenges, and candies can be delivered in a multi-phase mode. The compositions also contain buffer systems that facilitate oral absorption. A rapid release is followed by slower release of medicant(s) and dietary supplements. The buffer system is released simultaneously with the medicant(s) and dietary supplements, thereby facilitating transmucosal and buccal absorption of active ingredient(s). As a result, a substantial portion of medication(s) and dietary supplement(s) is absorbed quickly and is followed by slower absorption, thereby enhancing and prolonging delivery of desired ingredients to the bloodstream. The invention thus delivers, first, rapidly an initial pharmacologically effective dose of medicine and dietary supplements and, second, a prolonged pharmacologically sufficient dose for longer-term relief of symptoms or provision of therapeutic effect.

2. Description of the Prior Art

Most oral delivery systems for medications and dietary supplements involve swallowing with subsequent absorption of the active ingredient(s) through the gastrointestinal (GI) system. Although this can be an effective means of delivery of many medications and dietary supplements, oral administration generally involves consuming a capsule, powder or liquid with water. Most medications and dietary supplements are not well-absorbed in the stomach and must transit into the gut for complete absorption. Further, many medications and dietary supplements are subject to a “first-pass effect” during absorption. Active drug-metabolizing enzymes in the gut wall and in the liver can convert a large portion of medication(s) and dietary supplements to inactive and sometimes, active metabolites. Unabsorbed drug(s) and dietary supplements will pass through the GI system and be excreted. The active portion of the drug and dietary supplements that enter the bloodstream may represent only a small fraction of the amount of active ingredient originally present in the oral product. Consequently, more active ingredient than is needed is frequently placed in oral formulations to account for the losses that occur from metabolic inactivation during absorption and/or from excretion of unabsorbed active ingredient(s). Further, there is substantial inter-individual variability in absorption rates. Consequently, some individuals will absorb only sub-therapeutic amounts of medicine and dietary supplements, whereas other individuals may absorb therapeutic amounts and some may absorb more active ingredient than is needed. Also, transit through the GI system requires a substantial amount of time and the degree of absorption of active ingredients can be dependent upon numerous factors, including dissolution times, gastric emptying time, amount of liquid present, and influence of food upon absorption. The time-delay for production of effective blood levels of active ingredients by the oral route generally ranges from 30 minutes to four hours. Therefore, relief of symptoms from cravings, pain, disease, ailments and conditions by oral medications and dietary supplements is substantially delayed. Sometimes, the amount of active ingredient delivered to the bloodstream is ineffectual, whereas sometimes too much active ingredient is delivered and avoidable side-effects may develop. Furthermore, the individual who is taking the medication(s) and dietary supplements has virtually no control over the amount or speed of delivery of active ingredient once the product has been swallowed.

Many drugs and dietary supplements can be absorbed directly through the oral mucosa via buccal and sublingual routes. However, many active ingredients display chemical properties that prevent transmucosal absorption. All active ingredients can be classified by their chemical makeup as acids, neutrals or bases. Acids can be neutralized by basic chemicals to form corresponding salts that may display neutral or slightly basic characteristics. Bases can be neutralized by acidic chemicals to form corresponding salts that may display neutral or slightly acidic characteristics. Generally, the salt forms of active ingredients display greater water solubility and less lipid solubility than the corresponding unreacted form. Either the free, unreacted form or the salt form of active ingredients may be placed in the delivery vehicle for release into the mouth. In some cases, the active ingredient may be trapped or bound in either the ionized or unionized form in a vehicle, e.g., resins. Upon release from the delivery vehicle into the mouth, some active ingredients will be highly ionized as a result of the pH characteristics of oral fluids (saliva) while others will exist primarily in the neutral or unionized form. Once the active ingredient is released into the oral cavity, only the unionized form of the active ingredient(s) will be absorbed through the oral mucosa. The fraction of unionized active ingredient(s) that is present in saliva will determine the total amount that can be absorbed. We have found that the ratio of unionized active ingredient to ionized active ingredient in the mouth can be shifted to favor rapid and complete absorption in the mouth. This can be accomplished by release of buffer chemicals into the mouth from the delivery vehicle that alter the pH of saliva and subsequently, the amount of active ingredient in the unionized form that can be absorbed transmucosally. Unfortunately many existing gums, lozenges, candies, tablets, liquids and sprays contain no buffer chemicals, or do not adequately release sufficient amounts of buffer chemicals simultaneously with the drug to favor absorption of the unionized active ingredient.

Absorption of active ingredient(s) through the oral mucosa into the bloodstream effectively bypasses the degradation that occurs in the gut wall and liver and provides an alternate means of providing active ingredients. Consequently, higher bioavailability of active ingredients may be achieved by transmucosal delivery than by oral ingestion. An additional advantage of buccal/sublingual delivery of active ingredients is the rapid delivery of unmetabolized active ingredient(s) to the bloodstream. Under suitable pH conditions, active ingredients introduced into the oral cavity may be rapidly absorbed and can appear in the bloodstream within minutes of application. For many cravings, ailments, conditions and diseases, rapid relief provided by buccal/sublingual active ingredient delivery is highly preferred over the lag time introduced by swallowing these ingredients. An additional advantage provided by various embodiments of buccal/sublingual delivery systems of active ingredients according to the invention is the opportunity to control their rate of delivery. The individual user may either speed up delivery of the active ingredient or slow the release by enhancing or slowing dissolution of ingredients from the delivery system. Rapid release is usually accomplished by enhanced chewing, licking or sucking whereas slower release is accomplished by the opposite actions. Tablets, liquids and oral sprays offer the alternative of rapid-release of the entire dose together with buffers that allow immediate oral absorption.

Despite the various attempts in the art to incorporate active ingredients in a chewing gum, lozenge, candy, tablet or liquid, none have been satisfactory, in part, because of inadequate early release of active ingredient or inadequate control of pH conditions. Tablets, liquids and oral sprays have not sufficiently controlled the pH conditions of the mouth, thereby allowing too rapid absorption of medicant; consequently, much of the dose from oral sprays is ultimately swallowed and is exposed to the degradative processes of the gastrointestinal system.

The principal object of the present invention is to provide a delivery system for medicant(s) and dietary supplements in gums, lozenges, and candies that allows multi-phasic release of ingredients and buffer chemicals in the mouth, thereby providing the user with an effective early and sustained absorbable dose of ingredient(s). The tablet, liquid, and oral spray provides a means of immediate delivery of the medicant dose together with means for controlling pH of the mouth for rapid absorption of the medicant directly into the bloodstream.

### SUMMARY OF THE INVENTION

A transmucosal delivery system according to the invention comprises a carrier suitable for oral administration. A buffer is dispersed within the carrier, and there is sufficient buffer to achieve a predetermined pH within the oral cavity of a user. An active ingredient is dispersed within the carrier, at least a portion of the active ingredient being unionized at the predetermined pH for transmucosal absorption within the oral cavity.

The carrier is preferably one of a chewing gum, lozenge, candy, and a spray, all of which are suitable for oral administration to a human. For active ingredients that display basic characteristics, the buffer preferably elevates the pH of the oral cavity to more than 7, preferably to between a pH of between 7 and 10. For active ingredients that display acidic characteristics, the buffer preferably lowers the pH of the oral cavity to less than 7, preferably to between a pH of between 7 and 3 and more preferably between 5 and 4. A suitable buffer for basic active ingredients is potassium carbonate, although calcium and sodium based buffers may be used. A suitable buffer for acidic active ingredients is citric acid, although other buffers may be used. The active ingredient preferably achieves relief of symptoms, cravings, conditions, or like therapeutic effect. The active ingredient may be a medicament, dietary supplement, herbal product, or the like.

### DETAILED DESCRIPTION OF THE DRAWING

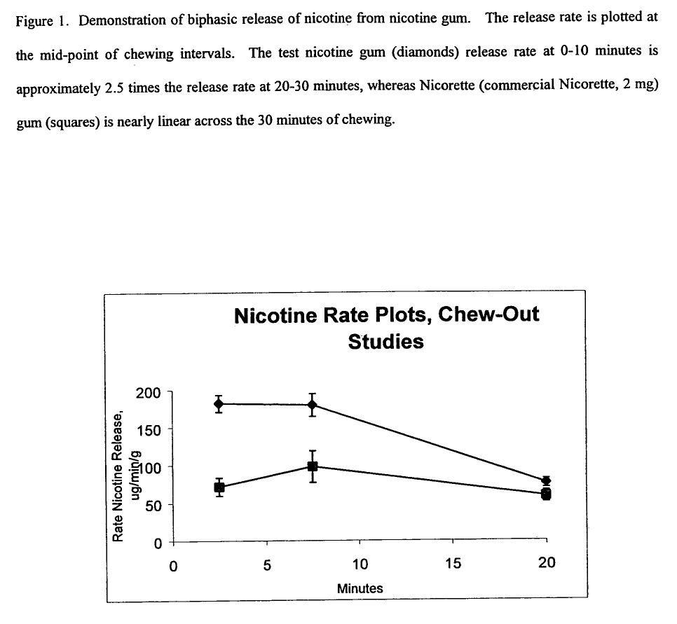

FIG. 1 is a graph containing data relating to the rate of release of nicotine versus time for a gum produced pursuant to the invention.

### DETAILED DESCRIPTION OF INVENTION

The present invention is a multi-phasic delivery system for medicant(s) and dietary supplements in gums, lozenges, and candies, a mono-phasic delivery of medicant(s) in a tablet, liquid, or oral spray and method for making those delivery systems. The delivery is multi-phasic because it is delivered at different dosing rates, via different forms of the active ingredient, or other than at a constant rate. For the gums, lozenges, and candies, the invention thus delivers, first, rapidly an initial pharmacologically effective dose of medicine and dietary supplement(s) and, second, a pharmacologically sufficient sustained dose for longer-term relief of symptoms, conditions or provision of therapeutic effect. The tablet, liquid, or oral spray invention delivers a rapid dose of pharmacologically effective medicant for immediate absorption. In certain embodiments, a sustained pharmacologically effective dose is thereafter released, preferably on an as needed basis. The invention employs chewing gum, lozenges, candies, tablets, liquids and sprays to achieve its purposes. The primary route for drug delivery is by the transmucosal route (sublingual, buccal, pharyngeal), although some minor amounts of active ingredient(s) may be ingested during chewing gum or sucking and wetting lozenges.

The present invention is a delivery system that delivers medicant(s) and dietary supplements into the oral cavity for subsequent absorption into the bloodstream in a highly efficacious manner. The speed of release of active ingredients is particularly important because a slow release rate would result in insufficient amount being absorbed into the bloodstream for relief of symptoms, conditions or cravings, whereas an extremely rapid release rate would result in unpleasant tastes and potential undesirable side effects from the active ingredient(s). In addition, an extremely rapid release rate would overwhelm the absorption process and result in swallowing of significant amounts of active ingredient(s), possibly producing gastric distress. Consequently, the ideal pattern of release of active ingredient(s) from gums, lozenges and candies is in the range of 10-60% percent by weight (“PBW”) of the total content of active ingredients within the first 10 minutes of placement into the oral cavity. The initial rapid release of active ingredients is followed by slower release of the remaining active ingredients over an additional period of 20-60 minutes that the delivery system remains in the mouth. The sustained relief thus further assures relief. The two doses help to prevent a relapse, a situation frequently- encountered where cravings, etc. are environmentally induced. Immediate dosing with the tablet, liquid or spray medicant allows rapid absorption of smaller amounts of medication, thereby avoiding swallowing excess drug and the accompanying gastric distress, while achieving essentially rapid relief of the cravings, etc.

Definitions

A. The term “medicant(s)” as used in this application, encompasses therapeutic substance(s) delivered by the invention to achieve a desired result. The term includes, but is not limited to, substances to relieve pain, ailments, disease, and/or infection.
B. The term “dietary supplements” as used in this application, encompasses vitamins, minerals, herbals and botanicals, herbal and botanical extracts, animal extracts, amino acids, proteins, concentrates, metabolites and constituents, teas, or other miscellaneous products.
C. The terms “gum”, “lozenge” and “candy” mean any substance chewed or dissolved in the mouth that provides medicine or dietary supplements transmucosally to the user. The term includes, but is not limited to, chewing gum or hard and soft candy-like substances.

The pattern of release of buffer chemicals likewise is important to the invention, because of the need to control oral pH. Many active ingredient components of medicants and dietary supplements that may be incorporated into the delivery system are sensitive to pH conditions. Medicants and dietary supplements that contain basic nitrogen moieties in their structure may demonstrate a pKa in the range of 3 to 11. Under acidic pH conditions in the mouth (pH 6.0 to pH 7.0), many of the useful compounds would be highly ionized and would not be efficiently absorbed into the bloodstream by the transmucosal route. For example, diphenhydramine, which has a pKa of 8.3 would be 50% ionized at a pH of 8.3. At lower pH conditions such as found in the mouth, diphenhydramine would be highly ionized and would be not be readily absorbed. Consequently, release of buffer chemicals must accompany release of medicants and dietary supplements that contain basic nitrogen moieties in their chemical structure. Buffer chemicals such as alkali carbonates rapidly elevate the pH of saliva in the mouth, and provide favorable pH conditions for efficient absorption of active ingredients. For example, use of potassium carbonate buffer in the delivery vehicle alters the mouth pH conditions to approximately 7-10 and provides a suitable environment for efficient absorption of most active ingredients containing basic nitrogen groups. Any one or combination of nontoxic potassium, sodium, calcium, magnesium or aluminum salts may be used to elevate mouth pH conditions. Examples of such chemicals are potassium carbonate, potassium bicarbonate, sodium carbonate, sodium bicarbonate, potassium citrate, sodium citrate, calcium carbonate, calcium phosphate, magnesium hydroxide, magnesium carbonate, magnesium trisilicate, aluminum carbonate and aluminum hydroxide.

Medicants and dietary supplements that contain acidic moieties in their structure may demonstrate a pKa in the range of 3 to 11. Under acidic pH conditions in the mouth (pH 6.0 to pH 7.0), many of the useful compounds would be highly ionized and would not be efficiently absorbed into the bloodstream. For example, phenobarbital, which has a pKa of 7.2, would be 50% ionized at a pH of 7.2. At pH conditions such as found in the mouth, phenobarbital would be substantially ionized and would be not be readily absorbed. By lowering the pH conditions of the mouth to 5.0 or less, less than 1% would be ionized and efficient absorption could occur. Consequently, release of buffer chemicals must accompany release of medicants and dietary supplements that contain acid moieties in their chemical structure. Non-toxic, physiologically-acceptable buffer chemicals such as organic acids rapidly lower the pH of saliva in the mouth and provide favorable pH conditions for efficient absorption of active ingredients. For example, use of citric acid in the delivery vehicle alters the mouth pH conditions to a pH<5.0 and provides a suitable environment for efficient absorption of most active ingredients containing acidic groups. Any one or combination of nontoxic acids may be used. Examples of such acids are citric, tartaric, fumaric, malic, maleic, gluconic, succinic, salicylic, adipic, phosphoric, benzoic, glutamic, sorbic, propionic, and tannic acid.

In general, according to the invention, chewing gum compositions containing medicant(s) and dietary supplements are comprised of a water insoluble chewing gum base portion, a water soluble portion that preferably includes sweeteners and active ingredient(s), fillers that may be insoluble or partially water soluble, and water insoluble flavorants and colorants. In addition, water soluble buffer chemicals are added to control the pH conditions in the mouth. In some cases, it may be desirable to include an antioxidant to protect the gum base, flavorants and active ingredients from oxidation.

Lozenges are flavored dosage delivery systems for medicant(s) and dietary supplements that are held in the mouth, wetted with saliva and sucked until dissolution occurs. Any of the various forms of lozenges, hard, boiled candy lozenges, or candies known in the art may be employed as a vehicle for medicant(s) and dietary supplements and buffer chemicals. A general discussion of lozenges forms of confectionery may be found in H. A. Lieberman, Pharmaceutical Dosage Forms, Volume 1: Tablets (1989), Marcel Dekker, Inc., New York, N.Y. at Medicated Confections, pages 419-582, which disclosure is incorporated herein by reference.

Tablets, liquids and sprays are dosage delivery systems for medicant(s) and dietary supplements that are placed in the mouth or under the tongue for rapid dissolution of active ingredient and absorption through epithelial tissues. A general discussion of tablet forms may be found in H. A. Lieberman, Pharmaceutical Dosage Forms, Volume 1: Tablets (1989), Marcel Dekker, Inc., New York, N.Y. at Medicated Confections, pages 75-418, which disclosure is incorporated herein by reference.

The initial rapid release of medicant(s) and dietary supplements, and buffer chemicals in the oral cavity from gum-based formulations occurs through the chewing actions and saliva dissolution of ingredients. The water soluble components begin to dissolve upon initiation of chewing due to the wetting action of saliva in the mouth. The insoluble materials of chewing gum (gum base, fillers, flavorants) primarily are retained in the mouth throughout the chewing period. Portions of water soluble medicant(s) and dietary supplements and buffer remain embedded in the gum base. In addition, a portion of the soluble active ingredient(s) may be reabsorbed into the gum base. After the initial rapid release of water soluble active ingredient(s) and buffer occurs, slower release of the remaining portion of water soluble active ingredient(s) and active ingredient(s) reabsorbed by the gum base occurs upon further chewing. The user can regulate the release of the soluble materials, including release of the active ingredient, by adjusting the rate of chewing.

To achieve a multi-phasic release profile of medicants from chewing gum, one should select medicants that are readily released from the gum matrix. The release of medicants from chewing gum is dependent upon physico-chemical factors of the medicant, upon properties of the gum base and upon properties of the buffer chemicals incorporated into the gum. Ideally, medicant(s) should be sufficiently water soluble to be released by the action of saliva and also should be sufficiently lipid-soluble to be absorbable through the oral mucosal tissues, i.e., buccal, sublingual, gingival and palatal tissues. Medicants of interest that display water-soluble properties generally contain polarizable chemical functional groups of the following types: a) amine groups; b) amide groups; c) carboxylic acid groups; d) phenolic groups; and e) alcohol groups. Medicants must also be sufficiently lipid-soluble to allow passage through the epithelial tissues of the oral mucosa. Consequently, medicants that display molecular weights greater than 100 daltons and contain functional groups such as aromatic rings, cycloalkyl rings, heterocyclic rings, and hydrocarbon chains are generally sufficiently water soluble and lipid soluble for efficient absorption through epithelial tissues. In addition to the chemical properties of the medicant, the gum base can influence the profile of release of the medicant. Gum bases that show a high affinity for the medicant(s) would not readily release sufficient portions of the medicant to provide a multi-phasic release pattern. Consequently, the gum base should be selected to allow efficient release of the medicant(s) within the first 10 minutes of chewing to provide a multi-phasic active ingredient release pattern. Numerous commercial gum bases may be employed to allow efficient release of the medicant within the first 10 minutes of chewing. Exemplary gum base formulations are disclosed in International Application No. WO 00/13662, entitled MEDICATED CHEWING GUM DELIVERY SYSTEM FOR NICOTINE, the disclosure of which is incorporated herein by reference. The buffer chemicals incorporated into the gum may also contribute to the rapid release of the active ingredient. For example, potassium carbonate buffer produces a slightly greater release of nicotine from gum compared to sodium carbonate. In addition, the buffer functions to increase the absorption of the active ingredient from saliva into the mucosal tissues. Consequently, the combination of the physical properties of the active ingredient, release-characteristics of the gum base, and selection of the appropriate buffer chemicals allow control over the amount of active ingredient initially released during the first ten minutes of chewing and of the amount of the active ingredient ultimately absorbed through the oral mucosa.

Following the initial rapid release of the active ingredients, the remaining portion of the active ingredient(s) in the gum will continue to be released at a slower rate. In like manner, the amount of the active ingredient remaining to be released will be determined by the above parameters. Generally, 10-50% PBW of the active ingredient(s) can be released within the first 10 minutes of chewing. Thereafter, the remaining 50-100% PBW of the active ingredient dose will be available for prolonged release,. The slower release of the active ingredient that occurs after the first 10 minutes of chewing likely occurs because of several factors. In cases where the active ingredient was present in an ionized salt form, the buffering action of saliva and the buffering action of the buffer chemicals transform the active ingredient from a salt form to a free-unionized form. This transformation is favorable and desirable for efficient absorption through the mucosal tissue, but also allows reuptake of the medicant into the gum base. Active ingredient that is reabsorbed by the gum base is substantially less water-soluble than the ionized form and will be released at a slower rate from the gum base (compared to the release rate of the water-soluble form) by continued chewing. Further, the facilitation of release of the active ingredient by the buffer chemical that occurs during the first ten minutes of chewing is no longer as effective. A substantial portion of the buffer is released during the first 10 minutes, therefore, there is less buffer to facilitate release of the active ingredient after the first 10 minutes. Consequently, after about 10 minutes, the remaining active ingredient will be released and absorbed at a slower rate. This multi-phasic profile of release is desirable by first providing an initial “bolus” release of the active ingredient during the first 5-10 minutes of chewing that will be rapidly absorbed into the bloodstream providing early symptom relief, and is followed by additional slower release of the active ingredient for sustaining blood levels and prolonging the effectiveness of the medicant.

Lozenges and medicated candies release active ingredients by dissolution in saliva. Dissolution times for lozenges and candies occur in the range of 3-30 minutes, preferably in the range of 5-15 minutes. Rapid release of medicant(s) and dietary supplements, and buffer occur initially from the lozenge because of stimulation of saliva flow by placement of the lozenge or medicated candy in the mouth. The enhanced saliva flow hastens lozenge dissolution thereby effecting enhanced release of active ingredients. As the initial rapid flow of saliva and enhanced dissolution rate of the lozenge begins to slow, there is slowed release of active ingredients.

Oral sprays provide an alternate means of rapid drug delivery to the oral cavity. The formulation is sufficiently buffered to allow immediate absorption of small medicant doses, thereby minimizing loss of drug from swallowing. The ease of administration of an oral spray conveniently allows for frequent additional doses to be administered on an as-needed basis for alleviation of symptoms and cravings.

The insoluble gum base material(s) utilized in gum formulation generally include both natural and synthetic elastomers and rubbers, natural and synthetic resins, fats, oils, waxes, softeners and inorganic fillers. The elastomers and resins may be selected from the many gum base materials known in the art including naturally-derived products such as chicle, julutong, and gutta percha and synthetic materials such as polyisobutylene, isobutylene, butadiene-styrene copolymers, polyethylene, polyvinylesters such as polyvinylacetate, and mixtures of any of the foregoing.

The gum base typically also includes other ingredients such as plasticizers and softeners, fats and oils, waxes, elastomer solvents, and filler materials. Such ingredients are well known in the art and are selected to adjust the gum base consistency to a desirable consistency for overall gum texture and chewability.

In one embodiment, it is highly preferable that the gum base be constructed to provide an initial soft chew that continues to be relatively soft-chewing throughout 30 to 45 minutes of chewing. The characteristics of a soft-chewing gum base facilitate the ability of the individual chewer to exert control over the amount and speed of release of active ingredient(s) during the chewing period. An additional desirable feature of the gum base is the ability to release reliably a portion of the active ingredient(s) during the early stages of chewing. Preferably, the gum base allows release of 10-50% of the initial dose of active ingredient(s) within the first 10 minutes of chewing.

The bulk sweeteners will constitute about 20-80% by weight of the chewing gum and may include both sugar and sugarless sweeteners. Such ingredients are well known in the art and are selected to impart improved palatability to the chewing gum and to aid in masking the bitter or unpleasant taste of some medicants and dietary supplements. High intensity sweetners may also be included such as saccharin and its various salts, cyclamic acid and its various salts, sucralose, and other high-intensity sweeteners known in the art.

In addition, flavorants may be used in the chewing gum within the range of 0.1-10% by weight, preferably between about 0.5-4% by weight of the chewing gum. The flavoring agents may include natural and synthetic agents and all such combinations thereof. Colorants may include food and pharmaceutical grade coloring agents.

A wide variety of lozenges and candies can also be used as the delivery vehicle for medicant(s) and dietary supplements and buffer chemicals. Generally, lozenges and candies have a base composed of a mixture of sugar and other carbohydrate bulking agents. Non-fermentable sugars such as sorbitol, mannitol, xylitol, isomalt and hydrogenated starch hydrolysates may also be used. A general discussion of lozenges and tablet forms of confectionery may be found in H. A. Lieberman, Pharmaceutical Dosage Forms, Volume 1: Tablets (1989), Marcel Dekker, Inc., New York, N.Y. at Medicated Confections, pages 419-582, which disclosure is incorporated herein by reference.

Oral sprays can be prepared in aqueous solution containing suitable buffers that provide appropriate conditions in the oral cavity for rapid absorption. In addition, suitable preservatives and solubilizing agents may be included to render the product stable for multiple administrations.

The chewing gum, lozenge candy, and spray formulations in this invention are suitable delivery vehicles for a broad array of active ingredients including medicant(s), dietary supplements, and buffer chemicals. The active ingredients may be used alone or in combination with other suitable active ingredients. The active ingredients may be used in their natural physical form, as coated microspheres, as coated solids, or as encapsulated solids.

By way of non-limiting examples, chewing gum, lozenge, candy, and spray formulations are particularly suited for transmucosal delivery of medicant(s) and dietary supplements, where rapid response is needed or desired and where the active ingredient(s) is metabolized or degraded by oral ingestion. Selected active ingredient(s) should exhibit suitable pharmacologic and physico-chemical properties including water solubility, lipid solubility, high pharmacologic potency, and stability, making them amenable to partial or complete transmucosal absorption.

Exemplary of the many categories of active medicants that are suitable for transmucosal delivery are anti-depressants, anti-diarrheals, anti-emetics, appetite enhancers, appetite suppressants, cough/cold medications, flu medicants, anti-diabetics, mental alertness enhancers, migraine/headache/fever medicants, anti-motion sickness medicants, nasal decongestants, analgesics, PMS medicants, sleep enhancers, sore throat medicants, and dietary supplements.

Exemplary of the many medicants suitable for transmucosal delivery are acetaminophen, amphetamine, aspirin, benzocaine, brompheniramine, buprenorphine, buspirone, butorphanol, caffeine, carbex, chlorpheniramine, clemastine, chromium, clotrimazole, cyclizine, cyclobenzaprine, dexbrompheniramine, dextromethorphan, dezocine, dibucaine, dihydroergotamine, dimenhydrinate, diphenhydramine, diphenoxylate, doxylamine, dyclonine, eldepryl, ephedrine, ergotamine, fentanyl, granisetron, guanifensin, hexobarbital, hydromorphone, hydroxyzine, ibuprofen, ketoprophen, levopromazine, levorphanol, lidocaine, loperamide, d-methamphetamine, d,l-methamphetamine, 1-methamphetamine, meclizine, menthol, methotimeprazine, miconazole, morphine, nalbuphine, naphazoline, naproxen sodium, naratriptan, nicotine, oxycodone, oxymetazoline, pentobarbital, peptide-medicants, pergolide, mesylate, pheniramine, phenobarbital, phentermine, phenylephrine, phenylpropanolamine, pilocarpine, promethazine, propylhexedrine, pseudoephedrine, protein-medicants, pyrilamine, rizatriptan, salagen, scopalamine, secobarbital, selegiline, sumatriptan, tetracaine, tetrahydrocannabinol, tramadol, triclosan, tripolidine, zolmitriptan, pentobarbital, hexobarbital, secobarbital, phenobarbital, and zolpidem.

Exemplary of the many dietary supplements suitable for transmucosal delivery are biotin, calcium, carnitine, choline, chromium, copper, creatine, fluorine, folate, inositol, iodine, iron, magnesium, manganese, molybdenum, niacin, niacinamide, pangamic acid (Vitamin B15), pantothenic acid, para-aminobenzoic acid, phosphorus, potassium, protein, riboflavin, selenium, silicon, thiamin, tin, vanadium, Vitamin A, Vitamin B1, Vitamin 2, Vitamin 3, Vitamin B6, Vitamin B12, Vitamin C, Vitamin D, Vitamin E, Vitamin K, and zinc,

Exemplary of the many herbal products also included as dietary supplements that are suitable for transmucosal delivery are bearberry, black cohosh, boldo, buckthorn bark, chamomile, Chinese ephedra, clove oil, cranberry, dandelion, echinacea, garlic, ginger, ginko biloba, goldenrod, horehound, horse chestnut, iceland moss, licorice, marshmallow root, milk thistle, nettle root, papaya, parsley, passion flower, plantain, sage, saw palmetto, senega snakeroot, slippery elm, St. John's Wort, thyme, tumeric, and valerian.

Various combinations of active ingredients may be incorporated into single delivery systems suitable for transmucosal delivery. For example, a cough/cold chewing gum could have a cough suppressant, e.g., dextromethorphan, an antihistamine, e.g., diphenhydramine, and a decongestant, e.g., pseudoephedrine, in the same preparation. Combinations of medicants, dietary supplements and herbal products could also be incorporated into single delivery systems suitable for transmucosal delivery.

A wide array of changes and modifications to the embodiments of the invention described above will be apparent to persons skilled in the art. The following examples are not intended to be construed as imposing limitations to the invention, but are presented to be illustrative of preferred embodiments.

### EXAMPLE 1

This Example illustrates a chewing gum composition of the present invention which contains a single medicant, nicotine. The amount of each ingredient used per 1 gram of gum is listed in Table I.

TABLE I

| Constituent | Weight | |
|---|---|---|
| Gum base | 600 | mg |
| Nicotine hydrogen tartrate | 6.5 | mg (base) |
| Potassium carbonate | 45 | mg |
| Sorbitol | 318.5 | |
| Spearmint flavoring | 24 | mg |
| Menthol | 6 | mg |

The chewing gum from the above composition had a soft, pleasant chewing consistency. When chewed for 30 minutes by seven individuals, the gum released approximately 63.5% of the nicotine. The peak pH produced in saliva as a result of buffer chemicals ranged from 8.8 to 9.3. In a clinical study of 28 individuals which compared the product to a commercial nicotine gum product (“Nicorette”) containing equivalent amounts of nicotine (2 mg of nicotine), significantly higher blood levels (p<0.001) were produced by the exemplary product compared to the commercial product at 5 minutes after the onset of chewing through 90 minutes (60 minutes post chewing).

The multi-phasic release pattern of medicant from gum is illustrated by a new nicotine gum formulated to have the desirable features of providing an initial rapid release of nicotine followed by a slower sustained release. As best shown in FIG. 1, the rate of release of nicotine (micrograms/mL/g gum) from the test gum was approximately three times higher during the first 5 minutes of chewing and was approximately two times higher during the 5-10 minute period of chewing. During the 10-30 minute chewing period, the release rate was approximately equal to Nicorette, 2 mg. In addition, it should be noted that the release rate by the nicotine test gum was approximately 2.5 times faster during the first ten minutes of chewing than the release rate of the test gum during the subsequent 10-30 minutes of chewing, clearly illustrating the multi-phasic release pattern of nicotine from the test nicotine gum. In contrast, the release rate of nicotine from the Nicorette gum was nearly constant across the entire period of chewing.

While this invention has been described as having a preferred embodiment, it is understood that the invention is not limited to the illustrated and described features. To the contrary, the invention is capable of further modifications, uses, and/or adaptations following the general principles of the invention and therefore includes such departures from the present disclosure as come within the known or customary practice in the art to which the invention pertains, and as may be applied to the central features set forth above, and which fall within the scope of the appended claims.

1-19. (canceled)

20. A transmucosal delivery system administrable in an oral cavity of a user, comprising:
a) a gum carrier suitable for oral administration;
b) a water soluble active ingredient dispersed within the gum carrier comprising at least one medicament selected from acetaminophen, amphetamine, aspirin, benzocaine, brompheniramine, buprenorphine, buspirone, butorphanol, caffeine, carbex, chlorpheniramine, clemastine, chromium, clotrimazole, cyclizine, cyclobenzaprine, dexbrompheniramine, dextromethorphan, dezocine, dibucaine, dihydroergotamine, dimenhydrinate, diphenhydramine, diphenoxylate, doxylamine, dyclonine, eldepryl, ephedrine, ergotamine, fentanyl, granisetron, guanifensin, hydromorphone, hydroxyzine, ibuprofen, ketoprophen, levopromazine, levorphanol, lidocaine, loperamide, d-methamphetamine, d,l-methamphetamine, 1-methamphetamine, meclizine, menthol, methotimeprazine, miconazole, morphine, nalbuphine, naphazoline, naproxen sodium, naratriptan, oxycodone, oxymetazoline, peptide-medicants, pergolide mesylate, pheniramine, phenobarbital, phentermine, phenylephrine, phenylpropanolamine, pilocarpine, promethazine, propylhexedrine, protein-medicants, pseudoephedrine, pyrilamine, rizatriptan, salagen, scopalamine, selegiline, sumatriptan, tetracaine, tetrahydrocannabinol, tramadol, triclosan, tripolidine, zolmitriptan, pentobarbital, hexobarbital, secobarbital, zolpidem, and combinations thereof; and
c) a water soluble buffer dispersed within the gum carrier, the buffer sufficient to achieve a pH within the oral cavity of the user for establishing multi-phasic delivery of the active ingredient, the multi-phasic delivery comprising first phase in which a first pharmacologically effective dose of the active ingredient is rapidly released at a first dosing rate upon initial oral manipulation of said gum carrier by a user, and a second phase in which a sustained second pharmacologically effective dose of the active ingredient is released at a second dosing rate upon subsequent oral manipulation of the gum carrier by the user for transmucosal absorption within the oral cavity, the first dosing rate being more rapid than the second dosing rate.

21. The system of claim 20, wherein from about 10 percent by weight to about 50 percent by weight of the medicament is released within ten minutes of the gum carrier having been administered to the oral cavity.

22. The system of claim 20, wherein the active ingredient is unionized at the pH for transmucosal absorption within the oral cavity.

23. The system of claim 20, wherein the buffer is basic and achieves a pH within the oral cavity of from about 7 to about 10.

24. The system of claim 23, wherein the buffer is selected from the group consisting of potassium carbonate, potassium bicarbonate, sodium carbonate, sodium bicarbonate, potassium citrate, sodium citrate, and combinations thereof.

25. The system of claim 20, wherein the buffer is acidic and is sufficient to achieve a pH within the oral cavity of from about 2.0 to about 4.5.

26. The system of claim 25, wherein the buffer is selected from the group consisting of citric, tartaric, fumaric, malic, maleic, gluconic, succinic, salicylic, adipic, phosphoric, benzoic, glutamic, sorbic, propionic, and tannic acid, and combinations thereof.

27. The system of claim 20, wherein:
the first phase is within ten minutes of the gum carrier having been administered to the oral cavity, during which from about 10 percent by weight to about 60 percent by weight of the active ingredient is released; and
the second phase extends over a period of 20 to 60 minutes after the first phase.

28. The system of claim 20, wherein:
the first pharmacologically effective dose lasts within ten minutes of the gum carrier having been administered to the oral cavity, during which from about 10 percent by weight to about 60 percent by weight of the active ingredient is released; and
the second pharmacologically effective dose extends over a period of 20 to 60 minutes after the first phase.

29. A transmucosal delivery system administrable in an oral cavity of a user, comprising:
a) a gum carrier suitable for oral administration;
b) a water soluble active ingredient dispersed within the gum carrier comprising at least one dietary supplement selected from biotin, calcium, carnitine, choline, chromium, copper, creatine, fluorine, folate, inositol, iodine, iron, magnesium, manganese, molybdenum, niacin, niacinamide, pangamic, acid (Vitamin B15), pantothenic acid, para-aminobenzoic acid, phosphorus, potassium, protein, riboflavin, selenium, silicon, thiamin, tin, vanadium, Vitamin A, Vitamin B1, Vitamin B2, Vitamin B3, Vitamin B6, Vitamin B12, Vitamin C, Vitamin D, Vitamin E, Vitamin K, zinc, and combinations thereof; and
c) a water soluble buffer dispersed within the gum carrier, the buffer sufficient to achieve a pH within the oral cavity of the user for establishing multi-phasic delivery of the active ingredient, the multi-phasic delivery comprising first phase in which a first pharmacologically effective dose of the active ingredient is rapidly released at a first dosing rate upon initial oral manipulation of said gum carrier by a user, and a second phase in which a sustained second pharmacologically effective dose of the active ingredient is released at a second dosing rate upon subsequent oral manipulation of the gum carrier by the user for transmucosal absorption within the oral cavity, the first dosing rate being more rapid than the second dosing rate.

30. The system of claim 29, wherein from about 10 percent by weight to about 50 percent by weight of the dietary supplement is released within ten minutes of the gum carrier having been administered to the oral cavity.

31. The system of claim 29, wherein the active ingredient is unionized at the pH for transmucosal absorption within the oral cavity.

32. The system of claim 29, wherein the buffer is basic and achieves a pH within the oral cavity of from about 7 to about 10.

33. The system of claim 32, wherein the buffer is selected from the group consisting of potassium carbonate, potassium bicarbonate, sodium carbonate, sodium bicarbonate, potassium citrate, sodium citrate, and combinations thereof.

34. The system of claim 29, wherein the buffer is acidic and is sufficient to achieve a pH within the oral cavity of from about 2.0 to about 4.5.

35. The system of claim 34, wherein the buffer is selected from the group consisting of citric, tartaric, fumaric, malic, maleic, gluconic, succinic, salicylic, adipic, phosphoric, benzoic, glutamic, sorbic, propionic, and tannic acid, and combinations thereof.

36. The system of claim 29, wherein:
the first phase is within ten minutes of the gum carrier having been administered to the oral cavity, during which from about 10 percent by weight to about 60 percent by weight of the active ingredient is released; and
the second phase extends over a period of 20 to 60 minutes after the first phase.

37. The system of claim 29, wherein:
the first pharmacologically effective dose lasts within ten minutes of the gum carrier having been administered to the oral cavity, during which from about 10 percent by weight to about 60 percent by weight of the active ingredient is released; and
the second pharmacologically effective dose extends over a period of 20 to 60 minutes after the first phase.

38. A transmucosal delivery system administrable in an oral cavity of a user, comprising:
a) a gum carrier suitable for oral administration;
b) a water soluble active ingredient dispersed within the gum carrier comprising at least one herbal product selected from bearberry, black cohosh, boldo, buckthorn bark, chamomile, Chinese ephedra, clove oil, cranberry, dandelion, echinacea, garlic, ginger, ginko biloba, goldenrod, horehound, horse chestnut, iceland moss, licorice, marshmallow root, milk thistle, nettle root, papaya, parsley, passion flower, plantain, sage, saw palmetto, senega snakeroot, slippery elm, St. John's Wort, thyme, tumeric, valerian, and combinations thereof; and
c) a water soluble buffer dispersed within the gum carrier, the buffer sufficient to achieve a pH within the oral cavity of the user for establishing multi-phasic delivery of the active ingredient, the multi-phasic delivery comprising first phase in which a first pharmacologically effective dose of the active ingredient is rapidly released at a first dosing rate upon initial oral manipulation of said gum carrier by a user, and a second phase in which a sustained second pharmacologically effective dose of the active ingredient is released at a second dosing rate upon subsequent oral manipulation of the gum carrier by the user for transmucosal absorption within the oral cavity, the first dosing rate being more rapid than the second dosing rate.

39. The system of claim 38, wherein from about 10 percent by weight to about 50 percent by weight of the herbal product is released within ten minutes of the gum carrier having been administered to the oral cavity.

40. The system of claim 38, wherein the active ingredient is unionized at the pH for transmucosal absorption within the oral cavity.

41. The system of claim 38, wherein the buffer is basic and achieves a pH within the oral cavity of from about 7 to about 10.

42. The system of claim 41, wherein the buffer is selected from the group consisting of potassium carbonate, potassium bicarbonate, sodium carbonate, sodium bicarbonate, potassium citrate, sodium citrate, and combinations thereof.

43. The system of claim 42, wherein the buffer is acidic and is sufficient to achieve a pH within the oral cavity of from about 2.0 to about 4.5.

44. The system of claim 42, wherein the buffer is selected from the group consisting of citric, tartaric, fumaric, malic, maleic, gluconic, succinic, salicylic, adipic, phosphoric, benzoic, glutamic, sorbic, propionic, and tannic acid, and combinations thereof.

45. The system of claim 38, wherein:
the first phase is within ten minutes of the gum carrier having been administered to the oral cavity, during which from about 10 percent by weight to about 60 percent by weight of the active ingredient is released; and
the second phase extends over a period of 20 to 60 minutes after the first phase.

46. The system of claim 38, wherein:
the first pharmacologically effective dose lasts within ten minutes of the gum carrier having been administered to the oral cavity, during which from about 10 percent by weight to about 60 percent by weight of the active ingredient is released; and
the second pharmacologically effective dose extends over a period of 20 to 60 minutes after the first phase.
